(19) United States
(12) Patent Application Publication
(10) Pub. No.: US 2026/0000911 A1
(43) Pub. Date: Jan. 1, 2026

(54) CANCER THERAPY DELIVERY SYSTEM AND COMPONENTS THEREOF

(71) Applicant: Boston Scientific Scimed, Inc., Maple Grove, MN (US)

(72) Inventors: Scott McGhee, Ottawa (CA); Kianna Y. Elahi-Gedwillo, Minneapolis, MN (US); Jonathan Nath, Inver Grove Heights, MN (US); Logan Ernster, Saint Paul, MN (US); Jacob Stoesz, Golden Valley, MN (US)

(21) Appl. No.: 19/254,836

(22) Filed: Jun. 30, 2025

Related U.S. Application Data
(60) Provisional application No. 63/666,467, filed on Jul. 1, 2024.

(51) Int. Cl. A61N 5/10; A61M 5/14; A61M 5/142

(52) U.S. Cl. CPC A61N 5/1007; A61M 5/1407; A61M 5/14212; A61N 2005/1021

(57) ABSTRACT

Embodiments herein relate to cancer therapy delivery systems including components thereof such as therapeutic mixture/suspension generating systems. In an embodiment, a cancer therapy suspension generating system is included having a fluid container defining an interior volume with a container sealing member sealing off an open end of the interior volume. A magnetic rotation member can be disposed within the interior volume and can be configured to rotate under the influence of a magnetic field. The magnetic rotation member can include a central hub, opposed arms that extend outward from the central hub, end lobes attached to ends of the opposed arms, and magnets disposed on or within the end lobes. The system can also include a fluid inlet and a fluid outlet. The system can also include a pump, wherein the pump pulls fluid out of the interior volume of the fluid container through the fluid outlet.

This application claims the benefit of U.S. Provisional Application No. 63/666,467, filed Jul. 1, 2024, the content of which is herein incorporated by reference in its entirety.

### FIELD

Embodiments herein relate to cancer therapy delivery systems including components thereof such as therapeutic mixture/suspension generating systems.

### BACKGROUND

According to the American Cancer Society, cancer accounts for nearly 25% of the deaths that occur in the United States each year. Cancerous tumors can form if one normal cell in any part of the body mutates and then begins to grow and multiply rapidly. Cancerous tumors can be a result of a genetic mutation to the cellular DNA or RNA that arises during cell division, an external stimulus such as ionizing or non-ionizing radiation, exposure to a carcinogen, or a result of a hereditary gene mutation. Regardless of the etiology, many cancerous tumors are the result of unchecked rapid cellular division.

Surgery is a common first-line therapy for many cancerous tumors. However, not every tumor can be surgically removed. Chemotherapy and immunotherapy are other common therapeutic approaches but can include substantial side effects. The use of radiation represents another approach. Specifically, radiation therapy aims at damaging the DNA of cancer cells so that they lose the capability to divide and proliferate, thus leading to the cell death process for the cancerous cells.

Brachytherapy is a form of radiation therapy where a sealed radiation source is placed inside or next to the area requiring treatment. As one form of brachytherapy, targeted radioembolization therapy can be used to treat unresectable tumors. For example, Y-90 glass microspheres can be delivered into or adjacent to a tumor through a microcatheter placed into an artery that supplies blood to the tumor. The beta radiation emitted by the Y-90 can exert a local radiotherapeutic effect on the tumor. Other radioisotopes can also be used in some types of brachytherapy.

### SUMMARY

Embodiments herein relate to cancer therapy delivery systems including components thereof such as therapeutic mixture/suspension generating systems. In a first aspect, a cancer therapy suspension generating system can be included having a fluid container defining an interior volume. A container sealing member can be configured to seal off an open end of the interior volume. A magnetic rotation member can be disposed within the interior volume and can be configured to rotate under the influence of an applied magnetic field. The magnetic rotation member can include a central hub, opposed arms that extend outward from the central hub, end lobes attached to ends of the opposed arms, and magnets disposed on or within the end lobes. The system can also include a fluid inlet and a fluid outlet, wherein the fluid inlet and fluid outlet pass through the container sealing member. The system can also include a pump, wherein the pump pulls fluid out of the interior volume of the fluid container through the fluid outlet.

In a second aspect, in addition to one or more of the preceding or following aspects, or in the alternative to some aspects, the central hub defines an open center aperture.

In a third aspect, in addition to one or more of the preceding or following aspects, or in the alternative to some aspects, the central hub can be a substantially toroidal structure.

In a fourth aspect, in addition to one or more of the preceding or following aspects, or in the alternative to some aspects, a ratio of the length of the magnetic rotation member to the inner diameter of the interior volume of the fluid container can be from 1:1.1 to 1:1.5.

In a fifth aspect, in addition to one or more of the preceding or following aspects, or in the alternative to some aspects, the cancer therapy suspension generating system can further include a rotation control base, the rotation control base can include a motor and a rotating bar, the rotating bar can include magnets, and a shaft, wherein the shaft conveys power from the motor to the rotating bar.

In a sixth aspect, in addition to one or more of the preceding or following aspects, or in the alternative to some aspects, can further include a control unit, wherein the control unit can be configured to rotate the magnetic rotation member at speeds of 300 to 500 RPM.

In a seventh aspect, in addition to one or more of the preceding or following aspects, or in the alternative to some aspects, the control unit can be configured to cause the motor to rotate in a first direction and then periodically switch to rotate in a second direction opposite the first direction.

In an eighth aspect, in addition to one or more of the preceding or following aspects, or in the alternative to some aspects, direction change timing can be from 200 to 1000 milliseconds.

In a ninth aspect, in addition to one or more of the preceding or following aspects, or in the alternative to some aspects, a height of the end lobes can be greater than the central hub.

In a tenth aspect, in addition to one or more of the preceding or following aspects, or in the alternative to some aspects, the fluid inlet can be configured to intake a gas to replace a volume of a fluid exiting the fluid outlet.

In an eleventh aspect, in addition to one or more of the preceding or following aspects, or in the alternative to some aspects, the cancer therapy suspension generating system can further include radioactive microspheres disposed within the interior volume, and a carrier fluid disposed within the interior volume.

In a twelfth aspect, in addition to one or more of the preceding or following aspects, or in the alternative to some aspects, the radioactive microspheres have a density of at least 3 times the carrier fluid.

In a thirteenth aspect, in addition to one or more of the preceding or following aspects, or in the alternative to some aspects, the pump can be a peristaltic pump.

In a fourteenth aspect, in addition to one or more of the preceding or following aspects, or in the alternative to some aspects, the fluid container can include a glass vial.

In a fifteenth aspect, in addition to one or more of the preceding or following aspects, or in the alternative to some aspects, the interior volume can have a volume of 10 to 200 milliliters.

In a sixteenth aspect, a cancer therapy suspension generating system can be included having a fluid container defining an interior volume, a container sealing member configured to seal off an open end of the interior volume, and a magnetic rotation member, wherein the magnetic rotation member disposed within the interior volume and configured to rotate under the influence of an applied magnetic field. The system can further include a fluid inlet passing through the container sealing member a fluid outlet passing through the container sealing member. The system can further include a pump that pulls fluid out of the interior volume of the fluid container through the fluid outlet. The system can further include a rotation control base including a motor and a rotating control member. The rotating control member can include perimeter uprights and a central cavity, wherein the fluid container can be configured to be suspended within the central cavity. The system can further include control magnets, wherein the control magnets can be attached to the perimeter uprights. A shaft can also be included that conveys power from the motor to the rotating control member.

In a seventeenth aspect, in addition to one or more of the preceding or following aspects, or in the alternative to some aspects, the magnetic rotation member can include a central hub and opposed arms, wherein the opposed arms extend outward from the central hub. The magnetic rotation member can further include end lobes, wherein the end lobes can be attached to ends of the opposed arms, and magnets, wherein the magnets can be disposed on or within the end lobes.

In an eighteenth aspect, in addition to one or more of the preceding or following aspects, or in the alternative to some aspects, the central hub can define an open center aperture.

In a nineteenth aspect, in addition to one or more of the preceding or following aspects, or in the alternative to some aspects, the central hub can be a substantially toroidal structure.

In a twentieth aspect, in addition to one or more of the preceding or following aspects, or in the alternative to some aspects, a height of the end lobes can be greater than the central hub.

In a twenty-first aspect, in addition to one or more of the preceding or following aspects, or in the alternative to some aspects, a ratio of the length of the magnetic rotation member to the inner diameter of the interior volume of the fluid container can be from 1:1.1 to 1:1.5.

In a twenty-second aspect, in addition to one or more of the preceding or following aspects, or in the alternative to some aspects, can further include a control unit, wherein the control unit can be configured to rotate the magnetic rotation member at speeds of 300 to 500 RPM.

In a twenty-third aspect, in addition to one or more of the preceding or following aspects, or in the alternative to some aspects, the control unit can be configured to cause the motor to rotate in a first direction and then periodically switch to rotate in a second direction opposite the first direction.

In a twenty-fourth aspect, in addition to one or more of the preceding or following aspects, or in the alternative to some aspects, wherein direction change timing can be from 200 to 1000 milliseconds.

In a twenty-fifth aspect, in addition to one or more of the preceding or following aspects, or in the alternative to some aspects, the fluid inlet can be configured to intake a gas to replace a volume of a fluid exiting the fluid outlet.

In a twenty-sixth aspect, in addition to one or more of the preceding or following aspects, or in the alternative to some aspects, the cancer therapy suspension generating system can further include radioactive microspheres, wherein the radioactive microspheres can be disposed within the interior volume, and a carrier fluid, wherein the carrier fluid can be disposed within the interior volume.

In a twenty-seventh aspect, in addition to one or more of the preceding or following aspects, or in the alternative to some aspects, the radioactive microspheres have a density of at least 3 times the carrier fluid.

In a twenty-eighth aspect, in addition to one or more of the preceding or following aspects, or in the alternative to some aspects, the pump can be a peristaltic pump.

In a twenty-ninth aspect, in addition to one or more of the preceding or following aspects, or in the alternative to some aspects, the fluid container can include a glass vial.

In a thirtieth aspect, in addition to one or more of the preceding or following aspects, or in the alternative to some aspects, the interior volume can have a volume of 10 to 200 milliliters

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic view of a cancer therapy delivery system in accordance with various embodiments herein.

FIG. 2 is a schematic view of a suspension generating system in accordance with various embodiments herein.

FIG. 3 is a schematic view of portions of a suspension generating system in accordance with various embodiments herein.

FIG. 4 is a schematic view of portions of a suspension generating system in accordance with various embodiments herein.

FIG. 5 is a schematic view of portions of a suspension generating system in accordance with various embodiments herein.

FIG. 6 is a perspective view of a magnetic rotation member in accordance with various embodiments herein.

FIG. 7 is a perspective view of a magnetic rotation member in accordance with various embodiments herein.

FIG. 8 is a perspective view of a magnetic rotation member in accordance with various embodiments herein.

FIG. 9 is a top plan view of a magnetic rotation member in accordance with various embodiments herein.

FIG. 10 is a schematic view of a rotation control base in accordance with various embodiments herein.

FIG. 11 is a schematic view of portions of a suspension generating system in accordance with various embodiments herein.

FIG. 12 is a schematic view of portions of a suspension generating system in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

In some cancer therapy approaches including the use of radioactive microspheres or other particulates, the microspheres are delivered to the patient through a catheter or other delivery line, carried along by a flow of carrier fluid and, once in the body, the microspheres or other particulates and the carrier fluid mix with blood flow and then the resulting mixture passes onto a target site in the body. Before passing through the catheter, the carrier fluid is mixed with microspheres forming a mixture or suspension that is then conveyed to the catheter. However, due to various factors, including a substantial difference in the density of the microspheres versus the fluid, the microspheres can settle out of the mixture or suspension relatively quickly, which makes forming the mixture or suspension challenging.

Embodiments of cancer therapy delivery systems herein and components thereof can thoroughly mix the carrier fluid and the microspheres and provide for more effective and consistent delivery of the microspheres. In some embodiments, a cancer therapy mixture/suspension generating system is included having a fluid container defining an interior volume and a container sealing member, wherein the container sealing member is configured to seal off an open end of the interior volume. A magnetic rotation member can be disposed within the interior volume and can be configured to rotate under the influence of an applied magnetic field. The magnetic rotation member can include a central hub, opposed arms extending outward from the central hub, and end lobes, wherein the end lobes are attached to ends of the opposed arms. Magnets can be disposed on or within the end lobes. The suspension generating system can include a fluid inlet passing through the container sealing member and a fluid outlet passing through the container sealing member. The suspension generating system can also include a pump, wherein the pump pulls fluid out of the interior volume of the fluid container and through the fluid outlet.

In other embodiments, a cancer therapy suspension generating system is included that can levitate a magnetic rotation member within the fluid container so that it does not contact the bottom of the fluid container as it rotates within the fluid container. For example, such a cancer therapy suspension generating system can include a fluid container defining an interior volume, a container sealing member configured to seal off an open end of the interior volume, a magnetic rotation member disposed within the interior volume and configured to rotate under the influence of an applied magnetic field, along with a fluid inlet and a fluid outlet both passing through the container sealing member. A pump is also included that pulls fluid out of the interior volume of the fluid container through the fluid outlet. In such embodiments, a rotation control base is also included. The rotation control base includes a motor and a rotating control member. The rotating control member includes perimeter uprights and defines a central cavity, wherein the fluid container is configured to be suspended within the central cavity. The rotating control member also includes control magnets attached to the perimeter uprights and a shaft that conveys power from the motor to the rotating control member. Aspects of these and other embodiments will now be discussed in greater detail.

The term “suspension” as used herein in reference to what embodiments of systems herein form and/or deliver as therapy to patients including carrier fluid and radioactive microspheres shall be used interchangeably with the term “mixture” unless the context dictates otherwise.

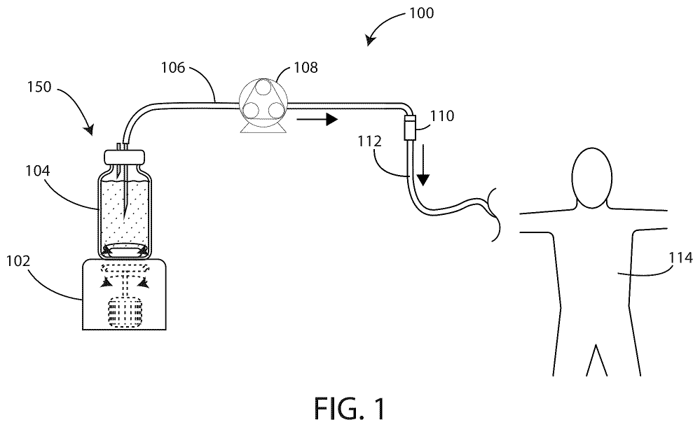

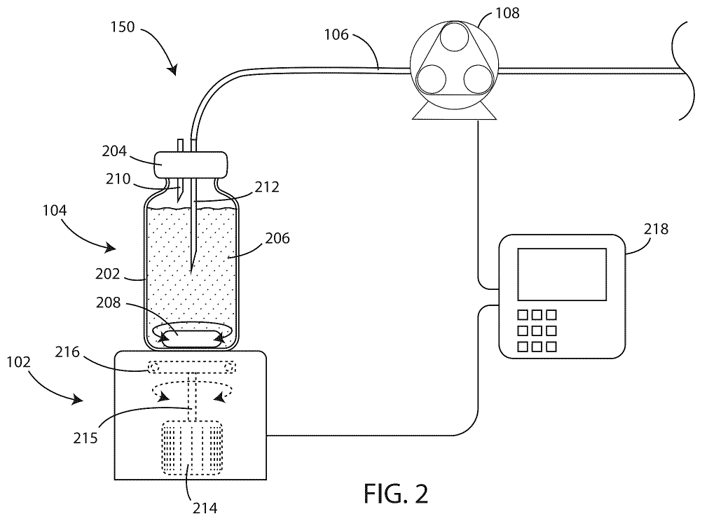

Referring now to FIG. 1, a schematic view of a cancer therapy system 100 is shown in accordance with various embodiments herein. The cancer therapy system 100 also includes a cancer therapy mixture/suspension generating system 150. The cancer therapy suspension generating system 150 includes a rotation control base 102, a fluid container 104 disposed over the rotation control base 102, a fluid line 106, and a pump 108. The cancer therapy system 100 also includes a fluid delivery catheter 112, which can be inserted into a patient 114. The fluid delivery catheter 112 includes a connection manifold 110. In operation, the rotation control base 102 provides a rotating magnetic field which acts upon a magnetic rotation member disposed within the fluid container 104 and causes the same to turn which mixes a carrier fluid and radioactive therapeutic microspheres together into a mixture or suspension. The pump 108 pulls fluid out of the fluid container 104 and into the fluid line 106 and conveys it to the fluid delivery catheter 112. While not shown in FIG. 1, in some embodiments a sensor (such as a radiation sensor, an optical particle counting sensor, or the like) can be included to confirm microsphere transfer to the fluid delivery catheter 112. In addition, in some embodiments, an additional fluid input can be provided to the pump 108 for a carrier fluid (such as saline) without microspheres, a contrast agent, or another composition. Further details of the system are provided with reference to FIG. 2.

FIG. 2 shows a schematic view of a suspension generating system 150 in accordance with various embodiments herein. As before, the suspension generating system 150 can include a rotation control base 102, a fluid container 104, a fluid line 106, and a pump 108.

The fluid container 104 can define an interior volume and a therapy suspension 206 can be disposed therein. In various embodiments, the therapy suspension 206 specifically includes radioactive microspheres and a carrier fluid. Details of exemplary microspheres are provided in greater detail below. The carrier fluid can be a biocompatible liquid such as an aqueous solution optionally including one or more salts or other components. The carrier fluid can be a saline solution in some embodiments.

The fluid container 104 can take various forms. In some embodiments, the fluid container 104 can be a vial 202, such as a glass vial. However, in other embodiments, the fluid container 104 can be formed of a ceramic, a polymer, a composite, a metal, or the like. The total volume of the fluid container 104 can vary. In some embodiments, the volume of the fluid container 104 can be from about 10 millimeters or less to about 200 or 300 milliliters or even more. The fluid container 104 can include a generally flat interior bottom.

The cancer therapy suspension generating system 150 also includes a container sealing member 204. The container sealing member 204 can be configured to seal off or plug an open end of the interior volume of the fluid container 104. In some embodiments, the container sealing member 204 can be formed of an elastomeric material, such as an elastomeric polymer. However, other non-elastomeric materials are also contemplated herein.

A fluid inlet 210 and a fluid outlet 212 can pass through the container sealing member 204. The fluid inlet 210 and fluid outlet 212 can be formed of various materials including, but not limited to, metals, glass, polymers, ceramics, composites, or the like. The fluid inlet 210 can be tubular in some embodiments and can be configured to intake a gas to replace a volume of a fluid that exits the fluid outlet 212, which can also be tubular, based on operation of the pump 108 pulling the mixture or suspension of microspheres and carrier fluid out of the interior volume of the fluid container 104. As such, the fluid inlet 210 can allow the interior of the fluid container 104 to remain at neutral pressure while fluid is removed therefrom. In some embodiments, the fluid outlet 212 can be approximately centered within the fluid container 104 and the fluid inlet 210 can be off to the side. However, various positions for both the fluid outlet 212 and the fluid inlet 210 are contemplated herein.

In many cases, the fluid inlet 210 can be configured to allow in air or another gas at a neutral pressure. However, in some embodiments, the fluid inlet 210 could be in fluid communication with a source of fluid that is pressurized, so that the interior of the fluid container is maintained at a positive pressure which could aid in microsphere entrainment into the fluid outlet. In some embodiments, the fluid inlet 210 can be used to introduce a liquid into the fluid container, such as additional volumes of a carrier fluid.

It will be appreciated that the pump 108 can be of various types and can be configured to operate at various speeds. Exemplary pump types can include, but are not limited to, peristaltic pumps, centrifugal pumps, positive displacement pumps, piston pumps, diaphragm pumps, and the like. However, in some embodiments the pump 108 can specifically be a peristaltic pump. In some embodiments, the pump 108 can be configured operate at various speeds including from about 3 milliliters/minute to about 20 milliliters/minute, or from 5 milliliters/minute to 7 milliliters/minute.

The cancer therapy suspension generating system 150 also includes a magnetic rotation member 208 disposed within the interior volume of the fluid container 104. The magnetic rotation member 208 can be configured to rotate within the interior volume of the fluid container 104 under the influence of an applied magnetic field which can be generated by the rotation control base 102.

The magnetic rotation member 208 can include magnets therein as described more fully below. The magnetic rotation member 208 can be formed of various materials including polymers, glasses, ceramics, and the like. However, in various embodiments herein the material chosen for the magnetic rotation member 208 can be one that is compatible with high-dose beta radiation, minimizes microsphere sticking, and resists degradation from contact with microspheres and/or interior surfaces of the fluid container. The magnetic rotation member 208 can take on various shapes as illustrated further below, but in some embodiments can include a central hub and a plurality of arms (such as two or more) extending outward therefrom.

The rotation control base 102 includes a motor 214, a shaft 215, and a rotating bar 216. When the motor 214 turns, the shaft 215 conveys power to the rotating bar 216, which includes magnets therein creating a rotating magnetic field that causes the magnetic rotation member 208 within the fluid container 104 to rotate. In some embodiments, the motor 214 can be linked to the rotating bar 216 through the shaft 215 such that each rotation of the motor 214 results in a rotation of the rotating bar 216, but in other embodiments, gearing or other hardware components can be included such that the ratio of rotations between the two components is not one to one. Thus, the magnetic rotation member 208 can be configured to rotate under the influence of an applied magnetic field provided by the rotating bar 216. The motor 214 can be of various types, such as various types of electric motor. In some embodiments, the motor 214 can specifically be a brushless electric motor and can be controlled by other components herein. In some embodiments, the motor 214 can be a stepper motor to enable precision control of spin rate, pauses, and reversing direction.

The cancer therapy suspension generating system 150 also includes a control unit 218. In various embodiments, the control unit 218 can control the motor 214, including controlling the speed of the motor 214 along with the direction of rotation. The control unit 218 can be in electrical communication with the motor and can include various components for controlling the same. In some embodiments, the control unit 218 can include a controller circuit. In some embodiments, the controller circuit can include a microcontroller to perform operations herein including controlling one or more motors, receiving and/or sending communications, managing a power supply circuit, receiving user inputs or other control commands, driving a display output, and the like. In some embodiments, the controller circuit can include a microprocessor. The microprocessor system can include components such as an address bus, a data bus, a control bus, a clock, a CPU, a processing device, an address decoder, RAM, ROM and the like. In some embodiments, the controller circuit can include an application specific integrated circuit-ASIC. In some embodiments, the control unit 218 can include a nonvolatile memory. In some embodiments, the non-volatile memory can be configured to store data herein. In some embodiments herein, components can be configured to communicate over a network (via wired or wireless means), such as the internet or a similar network.

While not shown in FIG. 2, in some embodiments, the system can also include a weight sensor such as to measure a weight of the fluid container and the contents thereof in order to provide verification of or feedback on the volume/weight of the microsphere and carrier fluid mixture/suspension that is delivered to the fluid delivery catheter.

In various embodiments, the control unit 218 can be configured to cause a motor 214 to rotate in a first direction and then periodically switch to rotate in a second direction opposite the first direction. The timing of direction change can be predetermined, can be programmed into the system, can be determined dynamically, or the like. In various embodiments, the control unit 218 can be configured to provide direction change timing from 200 to 1000 milliseconds, or from 400 to 800 milliseconds, or from 600 to 750 milliseconds. In various embodiments, the control unit 218 can be configured to rotate a magnetic rotation member 208 at speeds of 300 to 500 revolutions per minute (RPM) or from about 350 to 450 RPM.

The speed of rotation of the magnetic rotation member 208 along with design aspects of the magnetic rotation member itself can influence the amount of shear generated within the fluid container. In some cases, a speed of rotation that is too high can lead to a liquid/gas interface that extends downward as far or farther than the bottom (or intake) of the fluid outlet 212. This is undesirable as it can lead to air/gas being drawn into the fluid outlet 212 instead of the mixture/suspension of carrier fluid and microspheres.

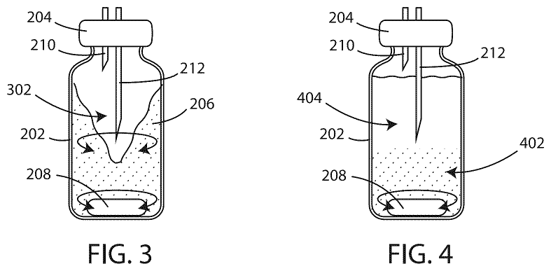

Referring now to FIG. 3, a schematic view of portions of a suspension generating system is shown in accordance with various embodiments herein. In this example, a fluid container is shown in the form of vial 202, which defines an interior volume including a therapy suspension 206 therein. The cancer therapy suspension generating system includes container sealing member 204, magnetic rotation member 208, fluid inlet 210, and fluid outlet 212.

The interior volume also includes an open headspace area 302. In this example, it can be seen that the therapy suspension 206 forms a vortex and the open headspace area 302 extends down far enough to cover an intake end of the fluid outlet 212. In this scenario, air from the open headspace area 302 can be drawn into the fluid outlet 212 which is undesirable. As such, a speed of rotation would desirably be less than a value creating the conditions shown in FIG. 3. In addition, a speed of rotation that is too high can result in the undesirable entrainment of air within the mixture/suspension of microspheres and carrier fluid.

Conversely, a speed of rotation that is too low can lead to undesirable separation of the carrier liquid and the therapeutic microspheres and/or insufficient microsphere suspension. Referring now to FIG. 4, a schematic view of portions of a suspension generating system is shown in accordance with various embodiments herein. As before, a fluid container is shown in the form of vial 202, which defines an interior volume including a therapy suspension 206 therein. The cancer therapy suspension generating system includes container sealing member 204, magnetic rotation member 208, fluid inlet 210, and fluid outlet 212.

The interior volume also includes a suspension region 402 along with carrier fluid only region 404. This separation is generally undesirable as it could lead to inconsistent amounts of microspheres in carrier fluid being drawn into the fluid outlet 212. This is particularly an issue in the case of mixtures/suspensions herein as the density of the microspheres (described below) is so much greater than the carrier fluid (which may be an aqueous solution and therefore have a density close to that of water).

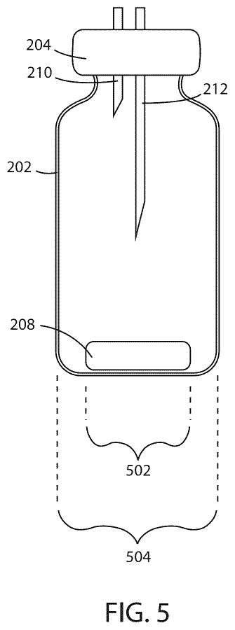

The size of the magnetic rotation member 208 relative to the inner diameter of the fluid container can impact shear near the interior walls of the fluid container. Referring now to FIG. 5, a schematic view of portions of a suspension generating system is shown in accordance with various embodiments herein. As before, a fluid container is shown in the form of vial 202, which defines an interior volume, as well as a container sealing member 204, magnetic rotation member 208, fluid inlet 210, and fluid outlet 212. The magnetic rotation member 208 includes a length 502, which can be from about 0.5 inches (12.7 mm) or less to 2 inches (50.8 mm) or more, or from about 0.75 inches (19.05 mm) to 1.25 inches (31.75 mm). The vial 202 includes an inner diameter 504 which can be larger than the length 502 of the magnetic rotation member 208. The inner diameter can be from about 0.6 inches (15.24 mm) or less to 2.1 inches (53.34 mm) or more, or from about 0.85 inches (21.59 mm) to 1.35 inches (34.29 mm). While not intending to be bound by theory, having a magnetic rotation member 208 with a length 502 that is relatively large relative to the inner diameter 504 of the vial 202 can aid in providing high levels of shear along the walls of the vial 202 and in the corners thereof. In some embodiments, the ratio of the length of the magnetic rotation member 208 to the inner diameter 504 of the interior volume can be from 1:1.1 to 1:2, or from 1:1.1 to 1:1.5, or from 1:1.1 to 1:1.5.

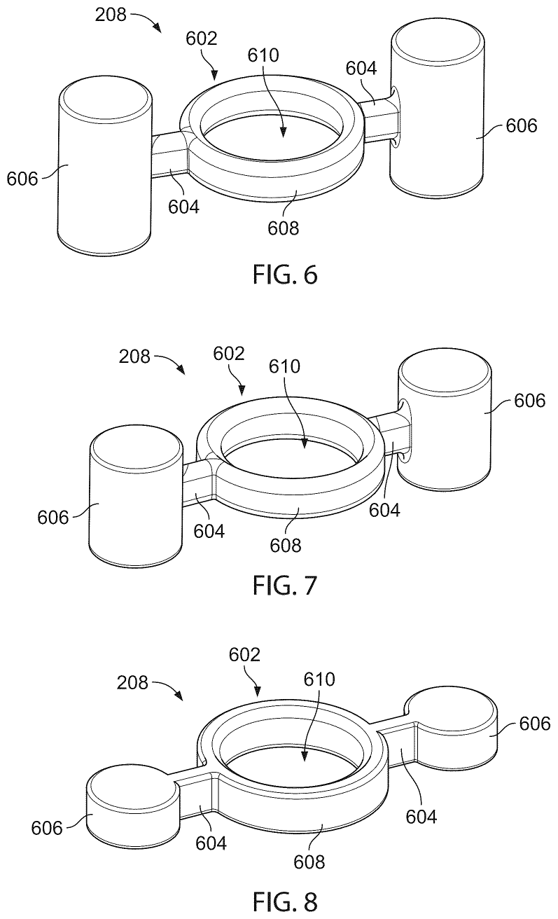

Magnetic rotational members herein can take various forms. Shapes of magnetic rotation members herein can minimize regions of low surface shear in the carrier fluid within the interior volume of the fluid container. Referring now to FIG. 6, a perspective view of a magnetic rotation member 208 is shown in accordance with various embodiments herein. The magnetic rotation member 208 includes a central hub 602, opposed arms 604, and end lobes 606. In this example, the central hub 602 includes a ring 608 and defines an open center aperture 610. While not intending to be bound by theory, the open center aperture can reduce or eliminate a low shear zone that may otherwise be present at the center of the magnetic rotation member 208. The diameter of the open center aperture 610 (or the inner diameter of the ring 608) can be at least about 50, 60, 70, 80, 90, or 95 percent of the outer diameter of the ring 608, or an amount falling within a range between any of the foregoing. In various embodiments, the central hub 602 can be a substantially toroidal shape. In some embodiments, portions of the ring 608 in cross-section can be substantially circular, ovoid, polygonal (and specifically square), irregular, or the like.

In various embodiments, the opposed arms 604 attach to and extend outward from the central hub 602. In many cases the opposed arms 604 can be centrally and/or symmetrically positioned so as to counterbalance one another during rotation. In many cases, there are two opposed arms 604. However, in other embodiments, there can be one, three, four, or more opposed arms 604. In cross-section, the opposed arms 604 can be substantially circular, ovoid, polygonal (and specifically square), irregular, or the like.

The end lobes 606 can be attached to the opposed arms 604, such as at the ends thereof. In some embodiments, the end lobes 606 can be substantially cylindrical. However, in other embodiments, the end lobes 606 can be spheroid, ovoid, polygonal, irregular, or the like. In various embodiments, the end lobes 606 can have a height that is greater than the central hub 602. However, in other embodiments, the end lobes 606 can have a height that is approximately equal to a height of the central hub 602. FIGS. 7 and 8 show alternative embodiments of the magnetic rotation member 208. In specific, FIG. 7 shows a magnetic rotation member 208 including a central hub 602, opposed arms 604, end lobes 606, ring 608, and open center aperture 610 as before. However, in FIG. 7, the height of the end lobes 606 is less than shown in FIG. 6. Further, FIG. 8 shows a magnetic rotation member 208 including a central hub 602, opposed arms 604, end lobes 606, ring 608, and open center aperture 610 as before. However, in FIG. 8, the height of the end lobes 606 is less than shown in both FIGS. 6 and 7.

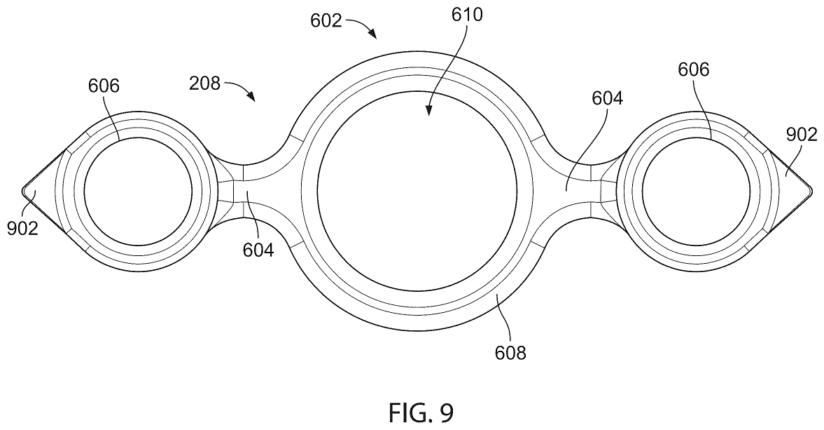

Various other features can be included with the magnetic rotation member 208. Referring now to FIG. 9, a top plan view of a magnetic rotation member 208 is shown in accordance with various embodiments herein. As before, the magnetic rotation member 208 includes a central hub 602, opposed arms 604, end lobes 606, ring 608, and open center aperture 610 as before. However, in the embodiment of FIG. 9, the end lobes 606 include an outer projection 902. In some embodiments, the outer projection 902 can narrow down to an outside most point. In some embodiments, the outer projection 902 can be roughly conical, pyramidal, or the like. While not intending to be bound by theory, the outer projection 902 can aid in reducing or eliminating low shear wall portions within the fluid container.

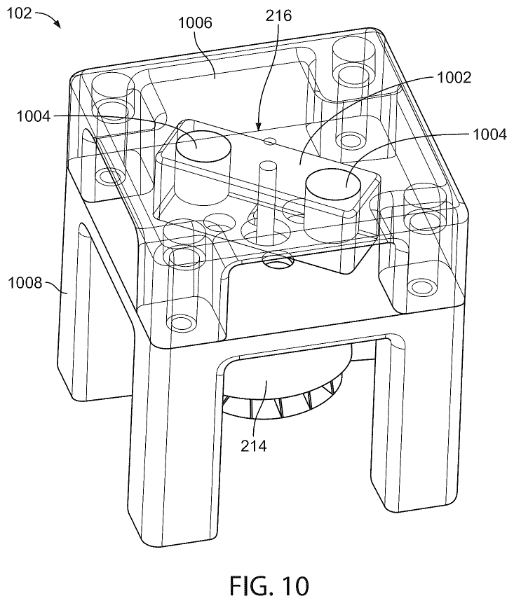

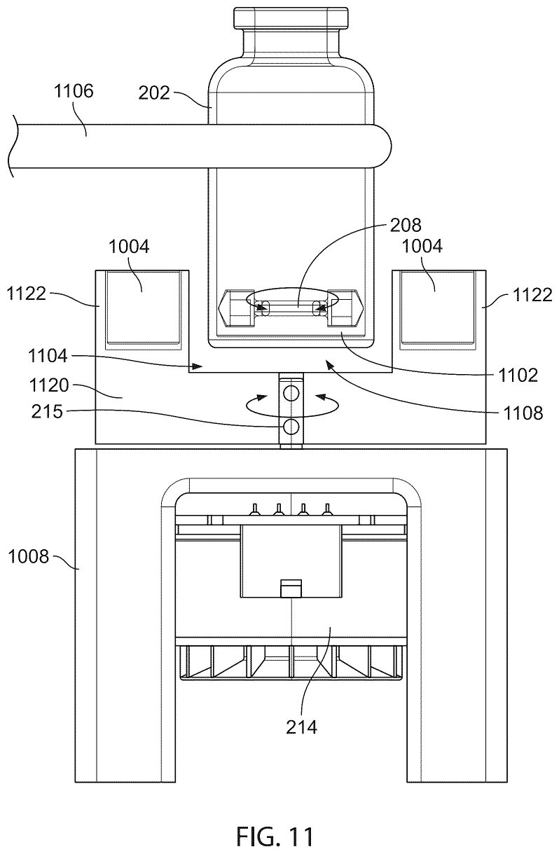

Rotation control bases can take on various forms herein. Referring now to FIG. 10, a schematic view of a rotation control base 102 is shown in accordance with various embodiments herein. The rotation control base 102 includes a motor 214 operably connected to a rotating bar 216. The rotating bar 216 includes a cross member 1002 and control magnets 1004. The rotation control base 102 also includes a platform 1006 and one or more support legs 1008. In operation, the fluid container can be disposed on or over the platform 1006 and power from the motor 214 can cause the rotating bar 216 to rotate generating a rotating magnetic field that can cause a central hub 602 disposed within the fluid container to rotate.

In some embodiments, cancer therapy suspension generating systems included herein can levitate a magnetic rotation member within the fluid container so that it does not contact the bottom of the fluid container as it rotates. This can be advantageous as there is less opportunity for catching microspheres between the bottom of the magnetic rotation member and the top surface of the bottom of the fluid container which in some scenarios could lead to breakdown of the microspheres into smaller particles.

Referring now to FIG. 11, a schematic view of portions of a mixture/suspension generating system is shown in accordance with various embodiments herein. As before, the rotation control base includes support legs 1008, a shaft 215, and a motor 214. However, in this example the rotation control base also includes a rotating control member 1120. The rotating control member 1120 includes a central cavity 1108. A fluid container, such as a vial 202, can be suspended within the central cavity 1108. For example, the cancer therapy suspension generating system can include a container support 1106 attached to the fluid container which can suspend the same within the central cavity 1108, such as separated from the bottom of the central cavity 1108 by a container air gap 1104. In some embodiments, the container support 1106 can be attached directly or indirectly to portions of the rotation control base that do not rotate such as the support legs 1008 thereof.

The rotating control member 1120 can include perimeter uprights 1122 to the outside of the central cavity 1108. The perimeter uprights 1122 can take various forms. In some embodiments, the perimeter uprights 1122 can take the form of columns, pillars, cylinders, or the like. In some embodiments, the perimeter uprights 1122 can even be integrated into a perimeter wall that surrounds the central cavity 1108.

The rotating control member 1120 includes control magnets 1004. The control magnets 1004 can create a magnetic field that both levitates the magnetic rotation member 208 by a levitation gap 1102 within the fluid container as well as causes rotation of the magnetic rotation member 208 within the fluid container.

In some embodiments, the orientation of the poles of control magnets 1004 as well as rotation member magnets 1210 can be arranged to provide for the levitation and rotation of the magnetic rotational member 208.

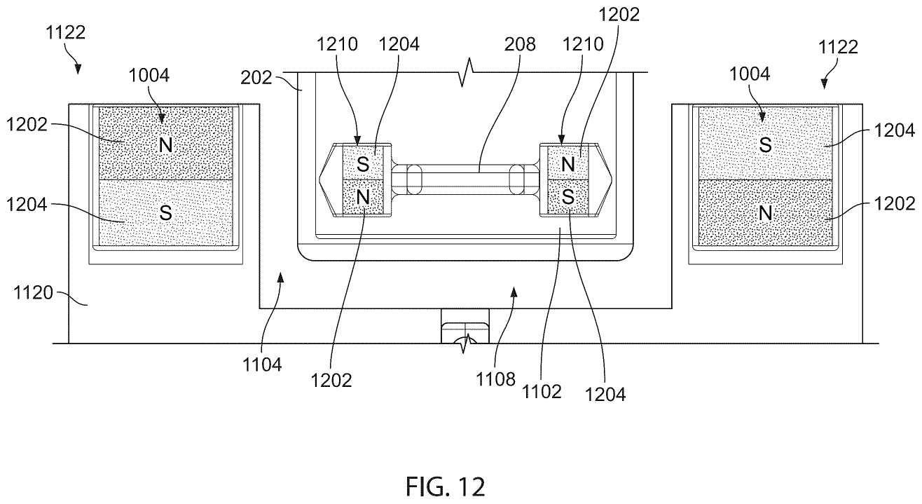

Referring now to FIG. 12, a schematic view is shown of portions of a suspension generating system in accordance with various embodiments herein. FIG. 12 shows a vial 202 suspended within the central cavity 1108 of a rotating control member 1120. Further, a magnetic rotation member 208 is within the vial 202 and separated from the bottom thereof by a levitation gap 1102.

Control magnets 1004 are attached to the perimeter uprights 1122 of the rotating control member 1120. In some cases, the control magnets 1004 are embedded within the perimeter uprights 1122. In this example, the control magnet 1004 on the left side is oriented such that it has its north magnetic pole 1202 at the top and its south magnetic pole 1204 at the bottom. In contrast, the control magnet 1004 on the right side is oriented such that it has its south magnetic pole 1204 at the top and its north magnetic pole 1202 at the bottom. These orientations are in opposition to the orientation of the rotation member magnets 1210 which, as illustrated, includes, for the rotation member magnet 1210 on the left, a south magnetic pole 1204 on the top and a north magnetic pole 1202 on the bottom whereas on the right, the south magnetic pole 1204 is on the bottom and the north magnetic pole 1202 is on the top. In this orientation, rotation of the rotating control member 1120 creates a rotating magnetic field that both causes rotation of magnetic rotation member 208, but also causes it to maintain the levitation gap 1102. It will be appreciated that the same effect can be achieved by reversing all of the north and south magnetic poles.

Various types of magnets can be used herein including both permanent magnets as well as, in some cases, electromagnets. Natural magnets herein can specifically include rare earth magnets. However, natural magnets used can include one or more of alnico, ferrite, neodymium, neodymium iron-boron, cobalt, samarium cobalt, and the like. While not intending to be bound by theory, rare earth magnets can be advantageous as they can enable highly controllable speed and direction changes.

### Microspheres

Microspheres herein can include those with a combination of yttria, alumina, and silica. By way of example, in some embodiments, microspheres herein can include Y₂O₃—Al₂O₃—SiO₂ in a 40:20:40 wt. % ratio. It will be appreciated however, that other types of microspheres are also contemplated herein.

In some embodiments, microspheres can be prepared by combining yittrium-89 with alumina and silica, in some cases also using a flame spheroidization method, and using neutron bombardment to convert Y-89 into the beta emitting radioisotope Y-90. In various embodiments, the amount of beta radiation can exceed 2500, 3000, 4000, 5000, 6000, 7000, 8000, or even 9000 Bq per sphere at the time of activity calibration (recognizing that the amount of radiation will drop after that point as the Y-90 radioisotope decays). In some embodiments, the microspheres can be provided in a vial with activity of 3 GBq or lower up to 20 GBq or higher (at calibration time or “reference date and time”). However, in some embodiments, the microspheres can be provided in a vial with activity of less than 3, 2.75, 2.5, 2.25, 2, 1.75, 1.5, 1.25, 1.0, 0.75, 0.5, 0.4, 0.3, 0.35, 0.2, 0.15, 0.1, 0.05, or 0.01 GBq, or less at calibration time, or an amount falling within a range between any of the foregoing.

It will be appreciated that dosages can vary based on factors including the type of tumor/tissue to be treated, location of the tumor/tissue to be treated, factors specific to a particular patient, and the like. In some embodiments the dosage of the therapy can be less than or equal to 5000 Gy, 4500 Gy, 4000 Gy, 3500 Gy, 3000 Gy, 2500 Gy, 2000 Gy, 1500 Gy, 100 Gy, 500 Gy, 400 Gy, 300 Gy, 250 Gy, 225 Gy, 200 Gy, 180 Gy, 150 Gy, 120 Gy, 100 Gy, 90 Gy, 80 Gy, 70 Gy, 60 Gy, 50 Gy, 40 Gy, 30 Gy, or 20 Gy, or an amount falling within a range between any of the foregoing.

The size of the microspheres can be extremely small. In some embodiments, the average diameter of the microspheres can be from about 20 micrometers (μm) to about 30 μm. However, in some embodiments the microspheres can be somewhat smaller or larger.

The density of the microspheres can be quite high. In some embodiments, the density of the microspheres can be above 3 g/mL, such as from 3.1 to 3.5 g/mL, or about 3.3 g/mL. By comparison, the density of water at room temperature is about 0.9978 g/mL. As such, the density of microspheres is much higher than an exemplary carrier fluid such as a saline solution which influences how readily such microspheres can settle out of a suspension.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A cancer therapy suspension generating system comprising:
a fluid container, the fluid container defining an interior volume;
a container sealing member, wherein the container sealing member is configured to seal off an open end of the interior volume;
a magnetic rotation member, wherein the magnetic rotation member is disposed within the interior volume and is configured to rotate under the influence of an applied magnetic field, the magnetic rotation member comprising
a central hub;
opposed arms, wherein the opposed arms extend outward from the central hub;
end lobes, wherein the end lobes are attached to ends of the opposed arms; and
magnets, wherein the magnets are disposed on or within the end lobes;
a fluid inlet, wherein the fluid inlet passes through the container sealing member;
a fluid outlet, wherein the fluid outlet passes through the container sealing member; and
a pump, wherein the pump pulls fluid out of the interior volume of the fluid container through the fluid outlet.

2. The cancer therapy suspension generating system of claim 1, the central hub defining an open center aperture.

3. The cancer therapy suspension generating system of claim 1, wherein the central hub is a substantially toroidal structure.

4. The cancer therapy suspension generating system of claim 1, wherein a ratio of the length of the magnetic rotation member to the inner diameter of the interior volume of the fluid container can be from 1:1.1 to 1:1.5.

5. The cancer therapy suspension generating system of claim 1, further comprising:
a rotation control base, the rotation control base comprising
a motor; and
a rotating bar, the rotating bar comprising magnets; and
a shaft, wherein the shaft conveys power from the motor to the rotating bar.

6. The cancer therapy suspension generating system of claim 5, further comprising a control unit, wherein the control unit is configured to rotate the magnetic rotation member at speeds of 300 to 500 RPM.

7. The cancer therapy suspension generating system of claim 6, wherein the control unit is configured to cause the motor to rotate in a first direction and then periodically switch to rotate in a second direction opposite the first direction.

8. The cancer therapy suspension generating system of claim 7, wherein direction change timing is from 200 to 1000 milliseconds.

9. The cancer therapy suspension generating system of claim 1, wherein a height of the end lobes is greater than the central hub.

10. The cancer therapy suspension generating system of claim 1, wherein the fluid inlet is configured to intake a gas to replace a volume of a fluid exiting the fluid outlet.

11. The cancer therapy suspension generating system of claim 1, further comprising:
radioactive microspheres, wherein the radioactive microspheres are disposed within the interior volume; and
a carrier fluid, wherein the carrier fluid is disposed within the interior volume.

12. The cancer therapy suspension generating system of claim 11, wherein the radioactive microspheres have a density of at least 3 times the carrier fluid.

13. A cancer therapy suspension generating system comprising:
a fluid container, the fluid container defining an interior volume;
a container sealing member, wherein the container sealing member is configured to seal off an open end of the interior volume;
a magnetic rotation member, wherein the magnetic rotation member is disposed within the interior volume and is configured to rotate under the influence of an applied magnetic field;
a fluid inlet, wherein the fluid inlet passes through the container sealing member;
a fluid outlet, wherein the fluid outlet passes through the container sealing member;
a pump, wherein the pump pulls fluid out of the interior volume of the fluid container through the fluid outlet;
a rotation control base, the rotation control base comprising
a motor; and
a rotating control member, the rotating control member comprising
perimeter uprights;
a central cavity, wherein the fluid container is configured to be suspended within the central cavity; and
control magnets, wherein the control magnets are attached to the perimeter uprights; and
a shaft, wherein the shaft conveys power from the motor to the rotating control member.

14. The cancer therapy suspension generating system of claim 13, the magnetic rotation member comprising:
a central hub;
opposed arms, wherein the opposed arms extend outward from the central hub;
end lobes, wherein the end lobes are attached to ends of the opposed arms; and
magnets, wherein the magnets are disposed on or within the end lobes.

15. The cancer therapy suspension generating system of claim 14, the central hub defining an open center aperture.

16. The cancer therapy suspension generating system of claim 14, wherein a height of the end lobes is greater than the central hub.

17. The cancer therapy suspension generating system of claim 13, wherein a ratio of the length of the magnetic rotation member to the inner diameter of the interior volume of the fluid container can be from 1:1.1 to 1:1.5.

18. The cancer therapy suspension generating system of claim 13, further comprising a control unit, wherein the control unit is configured to rotate the magnetic rotation member at speeds of 300 to 500 RPM.

19. The cancer therapy suspension generating system of claim 18, wherein the control unit is configured to cause the motor to rotate in a first direction and then periodically switch to rotate in a second direction opposite the first direction.

20. The cancer therapy suspension generating system of claim 19, wherein direction change timing is from 200 to 1000 milliseconds.
